(19) United States
(12) Patent Application Publication
(10) Pub. No.: US 2025/0302436 A1
(43) Pub. Date: Oct. 2, 2025

(54) Gastrointestinal motility capsule

(71) Applicants: Xiaoning Huai, Sunnyvale, CA (US); Tian Qiu, Jiangmen (CN)

(72) Inventors: Xiaoning Huai, Sunnyvale, CA (US); Tian Qiu, Jiangmen (CN)

(73) Assignee: Real Image Technology Co., Ltd, Shenzhen (CN)

(21) Appl. No.: 19/234,230

(22) Filed: Jun. 10, 2025

Related U.S. Application Data
(63) Continuation of application No. 19/183,786, filed on Apr. 19, 2025, now abandoned.
Application No. 19/183,786 is a continuation-in-part of application No. 19/171,272, filed on Apr. 6, 2025, now abandoned.
Application No. 19/171,272 is a continuation-in-part of application No. 19/097,894, filed on Apr. 2, 2025, now abandoned.
Application No. 19/097,894 is a continuation-in-part of application No. 17/520,669, filed on Nov. 7, 2021.
Application No. 17/520,669 is a continuation-in-part of application No. 17/372,518, filed on Jul. 12, 2021, now abandoned.

(30) Foreign Application Priority Data
Aug. 11, 2021 (CN) 2021109174009
Nov. 3, 2021 (CN) 2021112912234
Nov. 3, 2021 (CN) 2021112953022
Nov. 3, 2021 (CN) 2021226702773
Nov. 3, 2021 (CN) 2021226741119

(51) Int. Cl. A61B 8/12; A61B 8/00; A61B 8/08

(52) U.S. Cl. CPC A61B 8/12; A61B 8/08; A61B 8/5223

(57) ABSTRACT

A gastrointestinal motility detection capsule utilizes bidirectional ultrasound probe pairs to simultaneously obtain the distances from the capsule to the opposite sides of the gastrointestinal tract section. By adding the distances with the distance between the probe pair, a directional cavity diameter is obtained, which can eliminate data fusion errors caused by capsule movement. Based on multiple directional cavity diameters, gastrointestinal morphological features such as curvature, average diameter, volume, anatomical position, and 3D dynamics are acquired. Based on the time series of the features, gastrointestinal motility parameters such as peristaltic frequency and intensity, capsule movement trajectory, and emptying time are obtained. The capsule is to be used in routine gastrointestinal motility checks and disease diagnosis. The examinee does not need to clean the gastrointestinal tract and can pair it with different diets, making it convenient for self-examination at home or hospital check-ups.

### TECHNICAL FIELD

The invention relates to the technical field of gastrointestinal motility capsule.

### BACKGROUND

Gastrointestinal motility is crucial for human physiology and pathology, facilitating the movement, digestion, absorption, and expulsion of food. Traditional methods for measuring gastrointestinal motility, such as tracking radioactive markers, pose health risks due to radiation exposure, as noted in U.S. patent application Ser. No. 15/881,671. Consequently, there is a need for non-invasive, in vivo testing methods. Light, sound, and magnetism are commonly employed in non-invasive testing. Capsule endoscopy, while promising, often requires a clean digestive tract, which does not reflect normal physiological conditions. The development of 3D camera systems and gastrointestinal capsule robots with magnetic positioning, such as the Olympus EndoCapsule 10 system, has paved the way for this invention. Implementation technologies for general gastrointestinal capsule systems can be referenced in Chinese patent applications CN202011339489.7, CN202010260190.6, and U.S. Pat. No. 10,932,690B2. These capsule robots typically include sensors, controllers, and intelligent processors, with sensors and parts of the controller located within the capsule, while the intelligent processor is usually external. These components are connected via wired or wireless communication links. Given the widespread commercial application of capsule robots, their implementation is well-known to those skilled in the art and will not be elaborated upon in this description.

### SUMMARY OF THE INVENTION

The invention pertains to a capsule equipped with ultrasonic probes designed to gather data from a region of the digestive tract. This capsule, or an associated processor or apparatus, is capable of deriving morphological features of the region from the collected data. These features include, but are not limited to, curvature, volume, directional cavity diameter, inner diameter and equivalent inner diameter. The capsule includes a plurality of pairs of reversely positioned ultrasonic ranging probes which may form a spherical array. A first probe of a pair measures a distance to one side of a digestive tract wall and a second probe measures a distance to the opposite side. These measurements, along with the distance between the probes, are used to calculate the directional cavity diameter of the region. The probes operate with a directional deviation tolerance of less than 19 degrees and can function synchronously. The multiple probe pairs are set up to acquire various directional cavity diameters. By analyzing these diameters, the capsule or an associated processor can determine the principal axis of food transit, especially when the ratio of the largest to smallest diameter exceeds a certain threshold. This threshold varies depending on the digestive tract part but defaults to 2 if unspecified. Morphological features such as inner diameter, curvature, and volume can be derived from the cavity diameters. The inner diameter is calculated as the mean of diameters orthogonal to the principal axis or as the smallest diameter if the ratio is below the threshold. The capsule can also assess digestive motility parameters like peristaltic frequency and intensity, capsule movement trajectory, and transit time using time series data of these features. Furthermore, the capsule can determine anatomical positions within the digestive tract, including the stomach, by referencing morphological data. It can also register these positions with external coordinate systems. The capsule's shell is axially symmetrical, with more probe pairs oriented orthogonally to the long axis than parallel to it. The capsule contains a magnet for positioning the pose and position of the capsule, and the depth data of the inner wall of the digestive tract are collected in a target area or a region of concern in the digestive tract. Overall, this invention provides a comprehensive tool for analyzing the digestive tract's morphology and motility, offering valuable insights into digestive health and function.

The invention provides a first method for measurement of gastrointestinal motility, comprising the following steps:
obtaining one or more of depth, morphology and image data of the inner wall of digestive tract; obtaining data of the surface of the inner wall of digestive tract; extracting the morphological features of the surface data, wherein the morphological features include one or more of the anatomical parts of the inner wall of the digestive tract, curvature, inner diameter and volume.

The invention also provides a second method for measurement of gastrointestinal motility, comprising driving a capsule to a target area or region of concern of digestive tract and applying an intervene magnetic force on the capsule by a magnetron;
obtaining data of a first transit time of the capsule when no magnetic force is applied on the capsule;
obtaining data of a second transit time of the capsule when a first magnitude of a magnetic force is applied on the capsule and the difference between the first transit time and the second transit time is bigger than a threshold;
obtaining data of a second set of transit time of the capsule when the magnetic force is increased from the first magnitude to a second magnitude wherein transit of the capsule is blocked;
conducting an evaluation of gastrointestinal force based on the data of the first and second magnitude of the magnetic force, the first transit time and the second set of transit time, physical characteristics of the capsule and physical characteristics of gastric contents.

The invention provides a gastrointestinal motility measurement system based on a gastrointestinal capsule, which comprises a data acquisition module, a data processing module and a capsule. The data acquisition module and the data processing module are connected by a wired or wireless communication link. The data acquisition module is configured in the capsule, and comprises an ultrasonic distance measuring device or a camera for acquiring one or more of depth, morphology and image data of the inner wall of the digestive tract. The data processing module is preferably set in a control terminal outside the body, or in a distributed manner, wherein part of the functions is completed in the control terminal and part of the functions are completed in the capsule. The data processing module has at least one processor and at least one non-volatile storage medium, wherein the non-volatile storage medium contains instructions and parameters that can be read by the at least one processor, causing the at least one processor to run a digestive tract motility measurement program which coordinates the different modules. The data processing module is used to process the one or more of depth, morphology and image data to extract morphological features, including position, curvature, inner diameter and volume which are used as references for evaluation of gastrointestinal motility.

The invention provides another gastrointestinal motility measurement system, which comprises a control module, a magnetic driving module, a magnetic positioning module and a capsule. The control module, the magnetic driving module and the magnetic positioning module are connected by a communication link. The capsule is provided with a positioning magnet and a driving magnet, which could be a single magnet or two separate magnets. The positioning magnet generates a magnetic field signal, which is detected by the magnetic positioning module obtaining the position and motion data of the capsule in the digestive tract relative to an external coordinate system. The magnetic driving module generates a driving magnetic field, and the driving magnetic field acts on the driving magnet of the capsule to generate a driving magnetic force to drive the capsule to move in the digestive tract. The control module obtains a first position and motion data of the capsule under the action of gastrointestinal motility through a magnetic positioning module; obtains the second position and motion data of the capsule under the joint action of the gastrointestinal motility and the driving magnetic force. The gastrointestinal motility is estimated according to the first and second position and motion data and the driving magnetic force.

### BRIEF DESCRIPTION OF THE FIGURES

FIG. 1 is a schematic diagram of gastric peristalsis. It shows the morphological changes of gastric wall during peristalsis in the order from Step 1 to Step 4, wherein the inner diameter and curvature of the digestive tract flips between convex and concave during the gastrointestinal peristalsis.

FIG. 2 is an illustration of an ultrasonic capsule operation.

FIG. 3 is an illustration of the System Diagram.

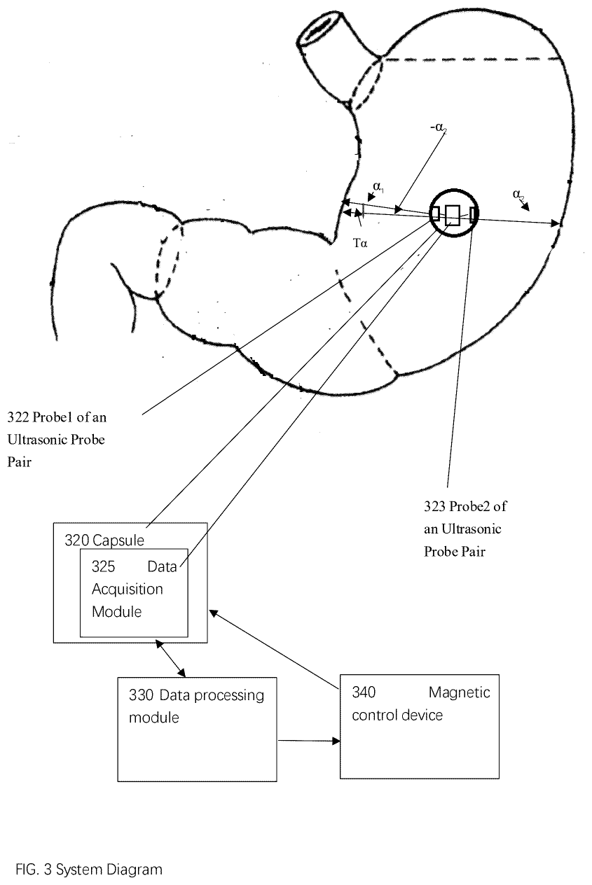

FIG. 4 is an illustration of the components in a spherical capsule.

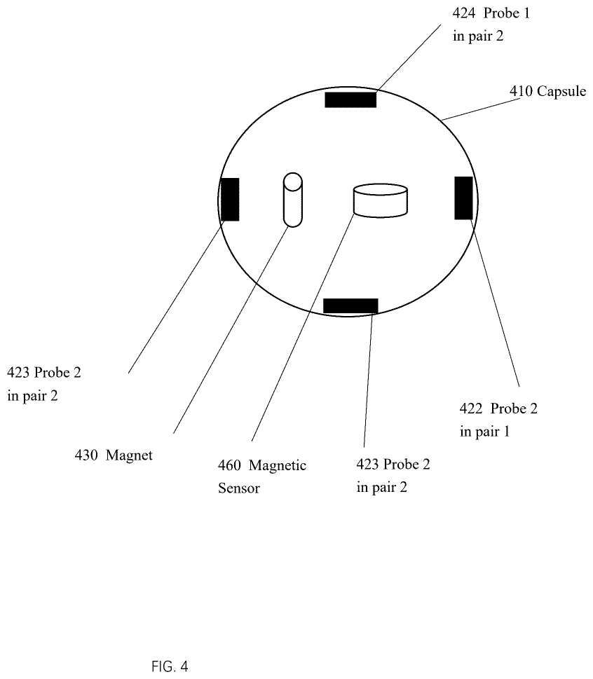

FIG. 5 is an illustration of spherical/oval Probe Array in the capsule of a spheroid shell.

FIG. 6 is an illustration of a different body shape for the capsule.

FIG. 7 is an illustration of obtaining inner diameters and transit time of the capsule

FIG. 8 is an illustration of registering the anatomical position with position data in an external system

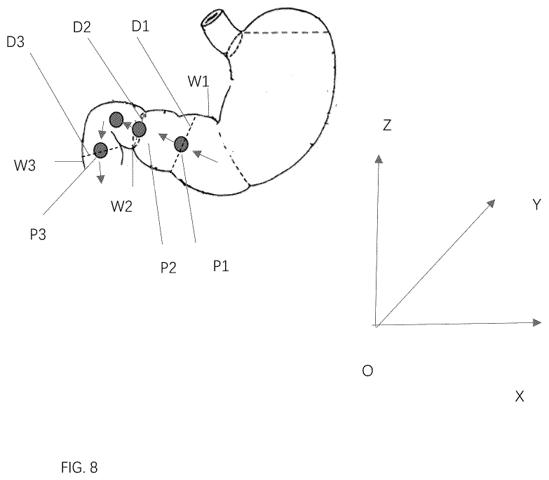

### DETAILED DESCRIPTION

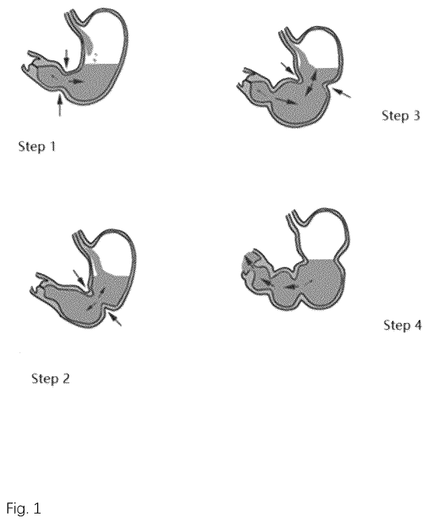

Gastrointestinal motility generally refers to the force and frequency of gastrointestinal contraction, relaxation and peristalsis under the action of gastrointestinal muscles. Its function is to make food move and be transmitted, so as to be digested, absorbed and emptied. An intuitive view of the relationship between the morphological characteristics of the digestive tract and the gastrointestinal motility comprises that under the action of the digestive tract muscles, the gastrointestinal peristalsis first produces deformation, including the change of the curvature of the digestive tract and the change of the inner diameter of the digestive tract. The deformation then transfers the force of the digestive tract muscle to the contents of the digestive tract, such as chyme, so as to make the contents of the digestive tract. Second, the digestive tract, like most other tissues in the human body, can be elastic. It is well known that the force on an elastic body is proportional to the deformation of the body under the force. Therefore, there is a close correlation between the morphological changes of the digestive tract and the gastrointestinal motility. As shown in FIG. 1, the changes in the inner diameter and curvature of the digestive tract include the frequency of the convex and concave flips of the surface of the inner walls of the digestive tract are directly correlated with the frequency and intensity of gastrointestinal peristalsis, which can be based on to determine the frequency and intensity of gastrointestinal motility. On the other hand, there are significant morphological differences in physiology and pathology of gastrointestinal peristalsis. For example, when stenosis, dilation or obstruction occurs, the normal rhythm of contraction and relaxation will change. Through statistical analysis of the data of the morphological characteristics and the changes of the morphological characteristics and the frequencies of the changes of regions of concerns of the digestive tract, a model of the morphological and dynamic characteristics of the digestive tract can be obtained, which can be used as a reference for evaluating the gastrointestinal motility. Like curvature and inner diameter, the morphological characteristics of digestive tract also include the change of volume of target areas or regions of concern of gastrointestinal lumen during peristalsis. The change of volume reflects the emptying amount of gastrointestinal peristalsis, which is related to the work done by gastrointestinal muscles and the energy produced.

The characteristic parameters of the digestive tract proposed above by the invention can preferably be acquired by first obtaining the depth map or point cloud of the inner wall of the digestive tract. Then the morphological features are extracted. Specifically, an ultrasonic distance measuring device can be preferably set in the capsule. After the capsule enters the body, the ultrasonic distance measuring device is started to obtain the distance from the capsule to the inner wall surface of the digestive tract. The ultrasonic measurement device can also collect the distance from the capsule to the multi-layer tissue structure of the inner wall of the digestive tract. Ultrasonic ranging mainly uses time difference ranging method. An ultrasonic probe emits directional ultrasonic wave and starts a timer at the same time of transmitting. The timer is stopped when the probe receives the reflected wave. Let V be the propagation velocity of the ultrasonic wave in the medium, T be the time difference between the transmitted wave and the returned wave recorded by the timer, and S be the distance from the transmitting point to the reflecting point

$$S = V \times T / 2$$

Let the capsule be of a sphere shape, the center of which is located at a point in the digestive tract lumen. The sum of the distance from the point to a point on the inner wall of the digestive tract in an arbitrary direction and the distance from the point to a point on the inner wall of the digestive tract in the opposite direction is defined as the directional cavity diameter of the digestive tract in the present invention. The directional cavity diameter is a measurement of the geometric size of the inner wall of the digestive tract by the ultrasonic ranging device, and also includes a pair of sampling points of the depth map of the inner wall of the digestive tract. There are multiple directional cavity diameters passing through any point. The spatial resolution of the depth map or point cloud and the final surface of the inner wall of the digestive tract is determined by the sampling interval, which conforms to the Nyquist law. A plurality of ultrasonic ranging probes can be preferably set in the capsule to form a spherical distribution ultrasonic ranging probe array platform including mechanism, circuit and control software, which is used to obtain multi-directional or panoramic depth map or point cloud data. Obviously, the denser the probe array, the more sampling points, and the higher the corresponding cost and circuit power consumption. Or a mechanical rotation device can be set on the platform of a sparse probe array, and it may rotate an angle after one sampling, and then conduct the next sampling. The platform comprises the following characteristics when conducting one measurement: First, all probes are located on a spherical surface; and second, the ranging directions of the two probes of any pair of probes are opposite yet correlated, and the connecting lines of the ranging directions of the two probes preferably pass through the ball center; thirdly, the measurements by two probes of a pair are synchronized, and fourthly, the ranging directions of a plurality of the directional cavity diameters are orthogonal if a number of the probe pairs is 2 or 3; wherein the ranging directions of at least 3 of the pairs are orthogonal if the number is larger than 3, wherein there is the third threshold of 19 degrees for deviation tolerance from orthogonality.

As the capsule is in a transit under the gastrointestinal peristalsis, the depth map or point cloud data from multiple sampling may preferably be matched, registered and fused. In addition to ultrasonic ranging device, 3D camera based on infrared or visible light sensor can also be used to obtain panoramic depth map or point cloud.

With the peristalsis of the alimentary tract, the capsule moves passively and randomly in the alimentary tract, and is finally discharged from the body. A preferred implementation of the invention can use the magnetic field generated by the magnetic control device to drive the capsule with a magnet in it to move in the digestive tract, or hold the capsule to stay in a target area or a region of concern for a measurement in-situ. Another preferred implementation of the invention is for the capsule to work intermittently, which is used to reduce the power consumption of the capsule battery.

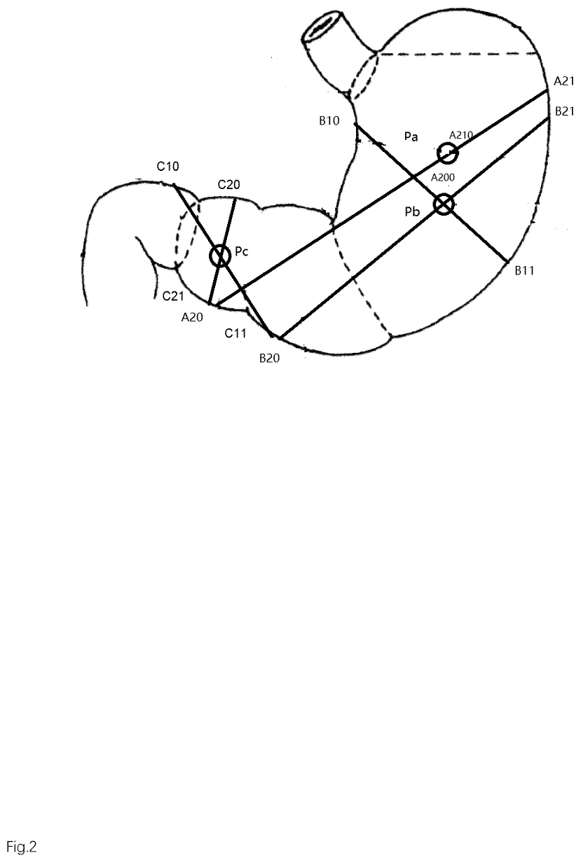

FIG. 2 is an illustration of an ultrasonic capsule operation. After the capsule enters a subject's body, it can get to a point Pa first. A line connecting two points on the gastric wall (A20, A21) intercepts with a circle representing a capsule at a point A210, and another point A200. The Probe1 of a probe pair located at A210 takes a measurement of the distance to point A21 on the gastric wall along the direction of (A210, A21) or (0, 9) in a spherical coordinate system with its coordinate origin set at Pa, wherein the distance is expressed by |a210, A21|. At the same time or in a synchronized manner, Probe 2 located at A200 on the opposite side of the capsule takes a measure of the distance between A200 to point A20 along the opposite direction (−θ, −φ), wherein the distance is expressed by |A200, A20|. Distance of |A210, A21|+|A200, A20|+|A200, A210| is a directional cavity diameter D passing through point Pa. Coordinates (θ, φ, |A210, A21|+½*|A200, A210|) and (−θ, −φ, |A200, A20|+½*|A200, A210|) are a pair of depth data obtained by the capsule at point Pa. In actual implementations, both the first direction and the second direction may deviate from the line as set above and result in for example α₁ and α₂ respectively. A deviation tolerance threshold is preferably provided as a margin for the system to work, wherein the angle between α₁ and −α₂ should be smaller than a threshold T_{α}, wherein T_{α} could be set at 19°.

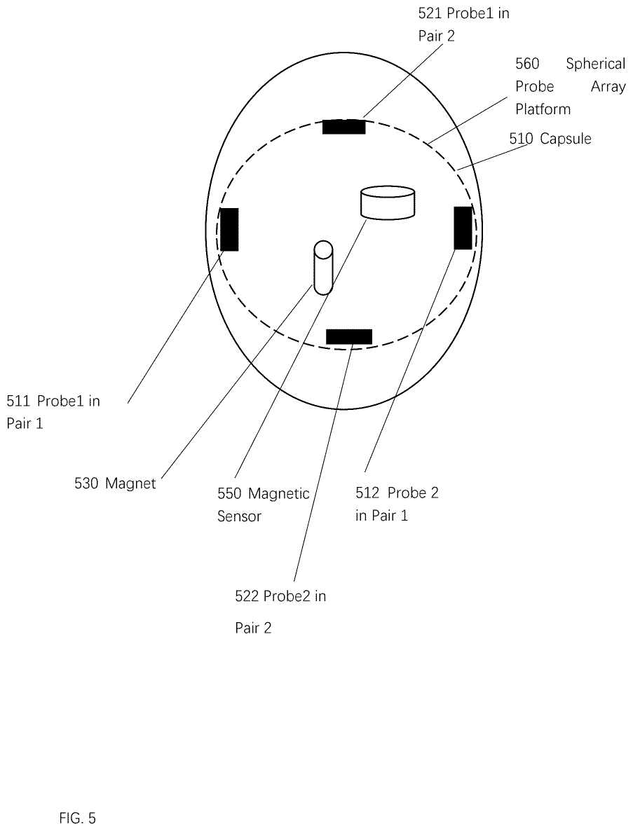

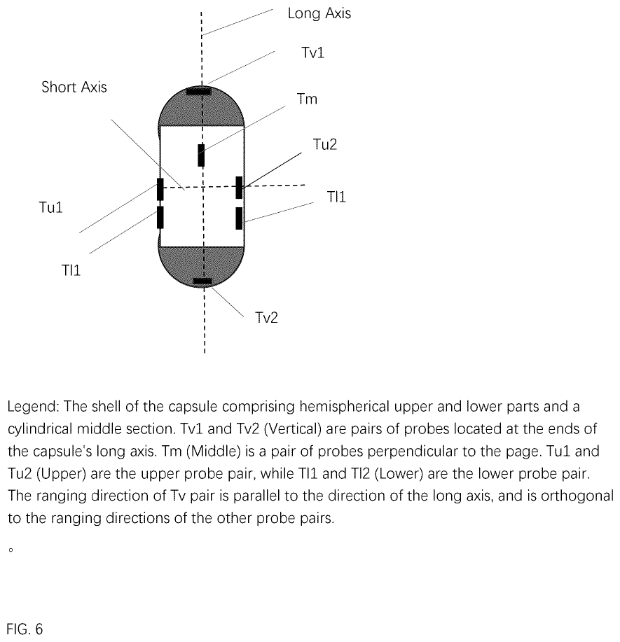

The collection of the depth data of all points of gastric wall acquired by the capsule at point Pa is the depth map at point Pa. The depth map obtained from different points, such as Pb, Pc, can be matched and fused into a depth map, and then the depth map can be transformed into a point cloud, or each depth map can be transformed into a point cloud, and then the point cloud can be matched and fused. Magnetic positioning may preferably be used to track and mark the pose and position of the capsule as a parameter for depth map or point cloud fusion. The point cloud can be regarded as a sample of the inner surface of digestive tract. Sparse point clouds can be smoothed and denoised by surface fitting algorithms to obtain surface data. With the peristalsis of the alimentary canal, the surface data of the inner wall of the whole alimentary canal can be accumulated. Because different parts of the human digestive tract have unique local morphological characteristics and corresponding relationship, the data processing module can recognize local morphological characteristics of the digestive tract through machine learning or other techniques. In an example to take a measure of a region of concern, such as a point Pc in FIG. 2, assuming the current position of the capsule being at a point Pa, the magnetic control device can be started to drive the capsule from point Pa to point Pc. When the magnetic positioning device confirms that the capsule has reached point Pc, the system control software of the data processing module starts the ultrasonic ranging device of the capsule to collect data. Furthermore, the data processing module will match the current pose and position data of the capsule collected in real time by magnetic positioning with the pose and position data obtained from analysis of the data of the inner wall of the digestive tract collected by the capsule to ensure the accuracy of the positioning. During a motility test, it may be optimized to minimize the perturbation of the test on the surrounding physiological environment, such as the design of the capsule of a small volume and with a round shape, a sleek shell of the capsule body, and a close density to that of chyme. In a test without intervention, the driving force of the magnetic control equipment can usually be in the zero state. In an intervention test, intervention force can be applied to maintain the capsule in a region of concern, or the capsule motion can be obstructed so he gastrointestinal force in the balance can be measured. As an embodiment, the capsule is observed at point Pc, near the pylorus. When the magnetic force reaches a first threshold, the transit time of the capsule increases. When the magnetic force reaches a second threshold, the capsule cannot be emptied. The peristaltic force of the capsule can then be estimated according to the transit time, the magnitude and direction of the magnetic force, the physical characteristics of the capsule and the physical characteristics of the gastric contents. After obtaining the depth map of the inner wall of digestive tract from the time series collected by the capsule, the data processing module can first convert the depth map into point cloud, and then perform surface fitting. Since the main function of the digestive tract is to move around the food, the direction of food motion can be regarded as the principal axis direction or the principal transit direction of the digestive tract. A statistical average value of a plurality of directional cavity diameters perpendicular to the principal axis at a point of concern in the digestive tract can be set as an inner diameter of the digestive tract at that point. The process of obtaining the average inner diameter is further explained with reference to FIGS. 5, 6, and 7. Tij represents the j-th probe of the i-th pair of probes (j=1, 2). At point A, the capsule synchronously acquires the cavity diameters in two orthogonal directions through probe pairs T1j and T2j, namely Ha1Ha2 and Va1Va2, where the longer Ha1Ha2 is the main channel direction measurement at point A. Due to the random movement of the capsule, its orientation is arbitrary. In larger sections of the digestive tract, such as the middle of the stomach at point A, the main channel direction measurement may not necessarily be the actual main channel direction. However, in smaller sections, especially where the length of the capsule's shell is close to or exceeds the actual inner diameter of that section, such as point B in the figure, there is a higher probability that the capsule's long axis aligns or coincides with the main channel direction of that section. Therefore, multiple probe pairs can be arranged in one or more planes perpendicular to the capsule's long axis, and the average of the cavity diameters measured by all probe pairs can be taken as the inner diameter of that section. Furthermore, the capsule can be configured in terms of dimensions, density, and center of gravity to increase the probability that its long axis aligns with the main channel of the digestive tract. Additionally, cavity diameters can be synchronously acquired in both the long and short axis directions, and their lengths can be compared to determine the capsule's orientation relative to the main channel direction at the moment of measurement. If the length ratio is greater than or equal to a threshold, the direction of the longest cavity diameter is taken as the main channel direction of that section, and the average of one or more cavity diameters orthogonal to the main channel direction is taken as the inner diameter of that section.

After obtaining the depth and/or morphological data of the digestive tract's inner wall from the time series collected by the capsule, the system control and processing module or the capsule's detection program can first convert the data into a point cloud and then perform surface fitting. Morphological feature extraction can be based directly on the original depth and/or morphological data or on the fitted surface data. Based on the surface data and the anatomical features of the digestive tract, the main channel direction at each point in the digestive tract can be estimated. Calculating surface curvature is a classic topic in differential geometry, with many algorithms available. Partial derivatives can be calculated on the data of the digestive tract's inner wall surface or on down sampled data of the surface. Different sections can have different curvature radius characteristics, and the curvature of surface data with different spatial frequencies corresponds to different curvature radii.

Volume calculation can be performed by selecting an adjustable-length line segment (L1, L2) along the main channel direction as the height, where L1 and L2 are the coordinates of the endpoints. Perpendicular planes S1 and S2 are drawn through L1 and L2 in the main channel direction. The closed body enclosed by planes S1, S2, and the surface data of the digestive tract's inner wall can be considered a volume at point Pc, and its calculation can use numerical integration methods. A simplified algorithm for volume calculation involves taking the product of three mutually orthogonal cavity diameters as the equivalent volume of that section, with an orthogonal tolerance of 38°. This is based on approximating the volume of a section of the digestive tract as a rectangular prism, on account of when using volume as a morphological feature to determine the anatomical position of the digestive tract, the focus is on relative size. The aforementioned algorithm provides good consistency across different sections, making it a feature with good discriminative power. Furthermore, if the three mutually orthogonal cavity diameters are r1, r2, and r3, then

r=(r1*r2*r3)\hat{ }(⅓) [4]

can be defined as an equivalent inner diameter of the digestive tract section.

The capsule's motion data, including displacement, velocity, and frequency, can be obtained through a magnetic positioning device. The rate of change and amplitude of the aforementioned digestive tract morphological features can be extracted from the time series of these features, and the frequency characteristics derived from morphological changes can be correlated with the frequency characteristics of the capsule's motion obtained from magnetic positioning.

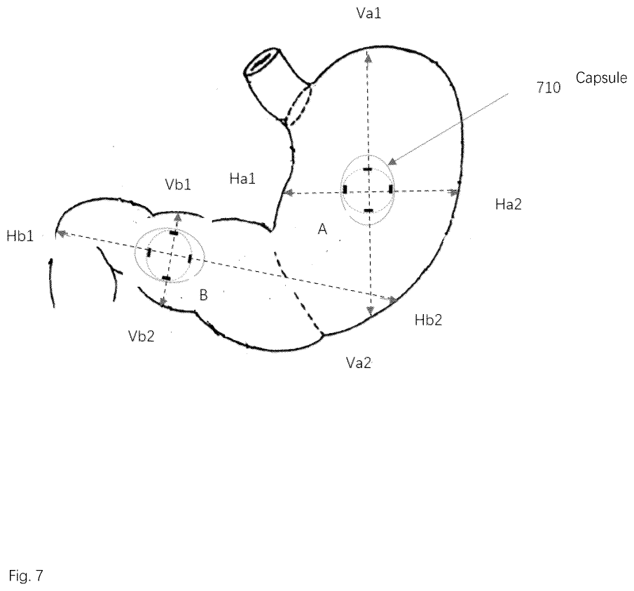

Further explanation of the capsule's acquisition of digestive tract motility parameters, including emptying time, based on the time series of morphological features is provided with reference to FIG. 7. The arrows in the figure represent the direction of food emptying, i.e., the main channel direction. The dashed lines D1, D2, and D3 represent the inner diameters detected by the capsule at digestive tract sections W1, W2, and W3, respectively. The XYZ coordinate system is located outside the digestive tract. P1->P2->P3 represents the capsule's motion trajectory detected by magnetic positioning at times t1, t2, and t3 (t3>t2>t1), with P1, P2, and P3 being the capsule's coordinates in this coordinate system, and P(t) being the operation function for obtaining the capsule's position through magnetic positioning, i.e., P(t1)=P1, P(t2)=P2, P(t3)=P3. D (t) is the operation function for obtaining the digestive tract's inner diameter by the capsule, i.e., D (t1)=D1, D (t2)=D2, D (t3)=D3. D1 is located at the gastric fundus, with the largest inner diameter (typically 6-7 cm in adults), D2 is located at the pylorus, with the smallest inner diameter (typically 1-2 cm in adults), and D3 is located in the duodenum, with an inner diameter between the two (typically 3-4 cm in adults), and

D1>D3>D2 [5].

Formula [5] and the corresponding statistical data express a set of anatomical correlations of the digestive tract's inner diameter near the pylorus, which is quite unique and can serve as a basis for determining the capsule's passage through the pylorus. Furthermore, the inner diameter D and the equivalent inner diameter r obtained from the aforementioned volume calculation [4] are correlated, and combining them can improve the accuracy of anatomical position recognition. Additionally, the W(t) function can represent a time series of the digestive tract's anatomical position, from which the capsule's emptying time at different anatomical positions can be directly obtained, such as the time through the pylorus, the small intestine, ileum, and colon.

That is, W(t1,t2,t3)=(W1 gastric fundus->W2 pylorus->W3 duodenum) [6].

Different foods or drugs can affect gastrointestinal motility. The above tests can be carried out in various food environment such as water, starch and wine.

1. A capsule, comprising ultrasonic probes, wherein a first probe ranges a first distance to a side of a wall of a region of a digestive tract along a first direction; a second probe ranges a second distance to an opposite side of the wall along a second direction; the first direction is opposite to the second direction with a direction deviation below a first threshold; wherein the capsule or a first processor or apparatus linked to the capsule obtains a directional cavity diameter by summing the first distance, the second distance and a distance between the two probes.

2. The capsule of claim 1, wherein the first threshold is set to 19 degrees, and the probes range in synchronization.

3. The capsule of claim 2, wherein the capsule or the first processor or apparatus uses directional cavity diameters to obtain morphological features comprising curvature or volume or inner diameter.

4. The capsule of claim 3, wherein the capsule or the first processor or apparatus is configured to acquire a surface of the wall; acquire a line segment (L1, L2) along a principal axis, wherein L1 and L2 are coordinates of endpoints of the line segment; obtain planes S1 and S2 vertical to the principal axis comprising L1, L2 respectively; obtain the volume enclosed by planes S1, S2 and the surface.

5. The capsule of claim 4, wherein the capsule or the first or a second processor or apparatus obtains a peristalsis frequency or peristalsis intensity referencing a spectral analysis of time series of the volume.

6. A capsule, comprising a plurality of ultrasonic probe pairs, wherein a first probe of each pair ranges a first distance to a side of a wall of a region of a digestive tract along a first direction; a second probe of the pair ranges a second distance to an opposite side of the wall along a second direction; the first direction is opposite to the second direction with a direction deviation below a first threshold; wherein the capsule or a first processor or apparatus linked to the capsule obtains a directional cavity diameter of the region by summing the first distance, the second distance and a distance between the two probes.

7. The capsule of claim 6, wherein the first threshold is set to 19 degrees; wherein the probes range in synchronization; wherein the plurality of the pairs orient mutually orthogonally if a number of the pairs is 2 or 3; wherein at least 3 of the pairs orient mutually orthogonally if the number is larger than 3, wherein orthogonality deviation is smaller than the first threshold.

8. The capsule of claim 7, wherein the capsule or the first processor or apparatus takes a length-to-width ratio of the largest directional cavity diameter over the smallest directional cavity diameter of the plurality of directional cavity diameters; selects ranging direction of the largest directional cavity diameter as principal axis if the ratio meeting or exceeding a second threshold, wherein the principal axis is a food transit direction in the region; wherein the second threshold is dependent on parts of the digestive tract or set to 2.

9. The capsule of claim 8, wherein the capsule or the first processor or apparatus uses the plurality of directional cavity diameters to obtain morphological features comprising inner diameter, or virtual inner diameter, or volume or virtual volume, or curvature; wherein the capsule or first processor or apparatus obtains, as the inner diameter, a mean value of a first set of directional cavity diameters with ranging direction orthogonal to the principal axis; or obtains the smallest directional cavity diameter as the inner diameter if the ratio is below the second threshold; wherein orthogonality deviation is smaller than the first threshold.

10. The capsule of claim 8, wherein the capsule or the first processor or apparatus takes a product of three mutually orthogonal directional cavity diameters as the virtual volume of the region; wherein the capsule or the first processor or apparatus obtains a cubic root of the virtual volume as the virtual inner diameter; wherein orthogonality deviation is smaller than the first threshold.

11. The capsule of claim 9, wherein the capsule or the first processor or apparatus is configured to determine an anatomical position of the region based on time series data of the morphological features referencing morphological correspondence of the digestive tract.

12. The capsule of claim 11, wherein the capsule or the first or a second processor or apparatus linked to the capsule through communication is configured to obtain, referencing the time series data of the morphological features or time series data of anatomical positions, at least one parameter of digestive motility comprising peristaltic frequency, or peristaltic intensity, or capsule movement trajectory, or capsule transit time.

13. The capsule of claim 12, wherein the capsule or the first or the second processor or apparatus is configured to obtain the capsule transit time or capsule movement trajectory referencing the time series data of the anatomical positions.

14. The capsule of claim 12, wherein the capsule or the first or the second processor or apparatus is configured to obtain the peristalsis frequency or peristalsis intensity of a stomach of the digestive tract referencing a spectral analysis of the time series data of inner diameters or virtual diameters of the stomach.

15. The capsule of claim 11, wherein the capsule or the first or second processor or apparatus is configured to register the anatomical position with position data of the capsule in an external coordinate system.

16. The capsule of claim 6, wherein a shell of the capsule is axially symmetrical; wherein a first number of a second set of the pairs that orient orthogonally to a longest symmetrical axis of the shell is more than a second number of a third set of the pairs that orient in parallel to the axis.

17. A capsule, comprising ultrasonic probes configured to obtain data of a region of a digestive tract; wherein the capsule or an apparatus or processor connected with the capsule obtains morphological features of the region based on the data; calculates a motility parameter of the digestive tract based on time series of the morphological features referencing correlation between the parameter and the morphological features.

18. The capsule of claim 17, wherein the morphological features comprising inner diameter, or virtual inner diameter, or volume or virtual volume, or curvature; wherein the parameter comprising peristaltic frequency, or peristaltic intensity, or capsule transit time.

19. The capsule of claim 17, wherein two probes of a pair are positioned in reverse orientations, each facing opposite sides of a wall within the region, ensuring that their ranging directions are aligned with a direction deviation below a first threshold, wherein the capsule, or a connected processor or apparatus, calculates a directional cavity diameter by summing measured distance from each probe to the wall and a distance separating the two probes; wherein the capsule comprising probe pairs configured to acquire a plurality of directional cavity diameters; wherein the capsule or processor or apparatus takes a ratio of the largest directional cavity diameter over the smallest directional cavity diameter of the plurality of the directional cavity diameters; selects ranging direction of the largest directional cavity diameter as principal axis if the ratio is bigger than or equal to a second threshold; obtains, as the inner diameter, a mean value of a first set of the directional cavity diameters with ranging direction orthogonal to the principal axis; or obtains the smallest directional cavity diameter as the inner diameter if the ratio is below the second threshold; wherein there is the first threshold for orthogonality deviation; wherein the second threshold is dependent on parts of the digestive tract or set to 2, wherein the first threshold is set to 19 degrees; wherein the principal axis is a food transit direction in the region.

20. The capsule of claim 19, wherein the capsule or the processor or apparatus obtains the peristalsis frequency or peristalsis intensity of a stomach of the digestive tract referencing a spectral analysis of the time series of inner diameters of the stomach.
